(19) United States
(12) Patent Application Publication
(10) Pub. No.: US 2010/0120760 A1
(43) Pub. Date: May 13, 2010

(54) BENZOFURAN-CARBOXAMIDE DERIVATIVES AS ANTIVIRAL AGENTS

(75) Inventors: Uwe Koch, Pomezia (Rome) (IT); Angela Claire Mackay, Pomezia (Rome) (IT); Frank Narjes, Pomezia (Rome) (IT); Ian Stansfield, Pomezia (Rome) (IT)

Correspondence Address: MERCK, P O BOX 2000, RAHWAY, NJ, 07065-0907, US

(73) Assignee: ISTITUTO DI RICERCHE DI BIOLOGIA MOLECOLARE P. ANG, POMEZIA (IT)

(21) Appl. No.: 12/594,855

(22) PCT Filed: Mar. 25, 2008
(86) PCT No.: PCT/GB08/50212

§ 371 Date: Oct. 6, 2009

(30) Foreign Application Priority Data
Apr. 12, 2007 (GB) 0707000.6

(51) Int. Cl. A61K 31/5377; C07D 307/78; C07D 405/14; C07D 261/08; C07D 413/14; C07D 249/08; A61P 31/12; A61K 31/343; A61K 31/4025; A61K 31/422; A61K 31/496; A61K 31/4196; A61K 31/4433

(52) U.S. Cl. 514/232.8; 549/458; 548/526; 548/249; 544/153; 544/378; 548/266.4; 546/284.1; 514/468; 514/422; 514/378; 514/254.11; 514/383; 514/338

(57) ABSTRACT

A compound of the formula (I): as defined herein, or a pharmaceutically acceptable salt thereof, or a pharmaceutical composition containing a compound of the formula (I) for use in inhibiting hepatitis C virus polymerase and/or of treating or preventing an illness due to hepatitis C virus,

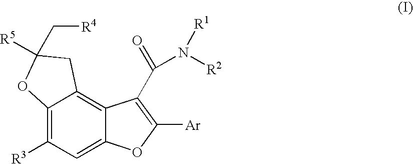

The present invention relates to novel dihydrofuranobenzofuran compounds, to pharmaceutical compositions containing them, to their use in the prevention and treatment of hepatitis C infections and to methods of preparation of such compounds and compositions.

Hepatitis C(HCV) is a cause of viral infections. There is as yet no adequate treatment for HCV infection but it is believed that inhibition of its RNA polymerase in mammals, particularly humans, would be of benefit.

Published International patent application WO 2004/041201 (Viropharma Incorporated and Wyeth) discloses the following benzofuran derivatives:

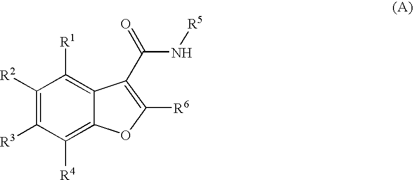

where R¹, R², R³, R⁴, R⁵, and R⁶ are defined therein, and their use for the treatment or prophylaxis of viral infections and diseases associated therewith, particularly those viral infections and associated diseases caused by the hepatitis C virus.

Published International patent application WO 2005/112640 (Viropharma Incorporated) discloses the following benzofuran derivatives:

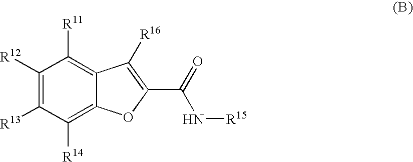

where R¹¹, R¹², R¹³, R¹⁴, R¹⁵ and R¹⁶ x are defined therein, and their use for the treatment or prophylaxis of viral infections and diseases associated therewith, particularly those viral infections and associated diseases caused by the hepatitis C virus.

Nevertheless, there remains the need to provide novel compounds for the prevention and treatment of viral infections.

It has been surprisingly found that certain dihydrofuranobenzofurans are inhibitors of the HCV NS5B RNA dependent RNA polymerase enzyme. These compounds are also inhibitors of HCV replication in tissue culture.

The present invention provides the compound of the formula (I):

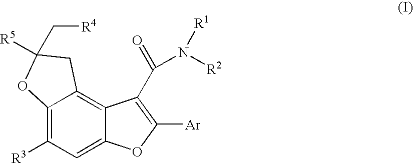

wherein

Ar is a moiety containing at least one aromatic ring and possesses 5, 6, 9 or 10 ring atoms, optionally containing 1, 2 or 3 heteroatoms independently selected from N, O and S, which ring is optionally substituted by groups Q¹ and Q²;

Q¹ is halogen, hydroxy, C₁₋₆alkyl, C₁₋₆alkoxy, (CH₂)₀₋₃aryl, (CH₂)₀₋₃heteroaryl, CONR^{c}R^{d}, (CH₂)₀₋₃NR^{c}R^{d}, O(CH₂)₀₋₃C₃₋₈cycloalkyl, O(CH₂)₁₋₃NR^{c}R^{d}, O(CH₂)₀₋₃CONR^{c}R^{d}, O(CH₂)₀₋₃CO₂H, O(CH₂)₀₋₃aryl, O(CH₂)₀₋₃heteroaryl, OCHR^{e}R^{f} or O(CH₂)₀₋₃ S(O)₂(CH₂)₀₋₃NR^{c}R^{d};

R^{c} and R^{d} are independently selected from hydrogen, C₁₋₆alkyl and C(O)C₁₋₆alkyl;

or R^{c} and R^{d}, together with the nitrogen atom to which they are attached, form a heteroaliphatic ring of 4 to 7 ring atoms, optionally containing 1 or 2 more heteroatoms independently selected from O and S and/or 1 or 2 groups independently selected from NH and NC₁₋₄alkyl, where said ring is optionally substituted by halogen, hydroxy, C₁₋₄alkyl or C₁₋₄alkoxy;

R^{e} and R^{f} are independently selected from hydrogen, C₁₋₄alkyl and C₁₋₄alkoxy;

or R^{e} and R^{f} are linked by a heteroatom selected from N, O and S to form a heteroaliphatic ring of 4 to 7 ring atoms, where said ring is optionally substituted by halogen, hydroxy, C₁₋₄alkyl or C₁₋₄alkoxy;

and where said C₁₋₄alkyl, C₁₋₄alkoxy and aryl groups are optionally substituted by halogen or hydroxy;

Q² is halogen, hydroxy, C₁₋₄alkyl or C₁₋₄alkoxy, where said C₁₋₄alkyl and C₁₋₄alkoxy groups are optionally substituted by halogen or hydroxy;

R¹ is hydrogen, C₁₋₆alkyl, C₂₋₆alkenyl, C₂₋₆alkynyl, (CH₂)₀₋₃C₃₋₈cycloalkyl or (CH₂)₀₋₃-phenyl;

R² is hydrogen or C₁₋₆alkyl;

R³ is hydrogen, halogen, hydroxy, C₁₋₆alkyl, C₂₋₆alkenyl, C₂₋₆alkynyl, C₃₋₈cycloalkyl, (CH₂)₀₋₃-phenyl, OC₁₋₆alkyl, O(CH₂)₀₋₃C₃₋₈cycloalkyl, O(CH₂)₀₋₃-phenyl, NR^{a}R^{b}, Het or heteroaryl, optionally substituted by C₁₋₄alkyl or C(O)C₁₋₄alkyl;

R^{a} and R^{b} are independently selected from hydrogen, C₁₋₆alkyl, C₁₋₆alkylene-OH and SO₂C₁₋₄alkyl;

R⁴ is hydrogen, halo, hydroxy, NR^{c}R^{d}, heteroaryl, O-heteroaryl, C(O)OC₁₋₄alkyl or C(O)NR^{c}R^{d}, optionally substituted by C₁₋₄alkyl, halo, hydroxy or oxo;

R^{c} and R^{d} are independently selected from hydrogen, C₁₋₄alkyl or aryl;

or R^{c} and R^{d}, together with the nitrogen atom to which they are attached, form a 5- or 6-membered heteroaliphatic ring optionally containing 1 or 2 more heteroatoms independently selected from O and S and/or 1 or 2 groups independently selected from S(O), S(O)₂, NH and NC₁₋₄alkyl;

R⁵ is hydrogen, C₁₋₆alkyl, C₂₋₆alkenyl, C₂₋₆alkynyl or (CH₂)₀₋₃cycloalkyl;

and pharmaceutically acceptable salts thereof.

In one embodiment of the present invention, Ar is a 5- or 6-membered aromatic ring optionally containing 1 or 2 heteroatoms independently selected from N, O and S, which ring is optionally substituted by Q¹ as hereinbefore defined. Preferably, Ar is phenyl, pyridyl, pyridazinyl, pyrimidinyl, pyrazinyl, thienyl, furanyl, pyrazolyl or imidazolyl, optionally substituted by Q¹ as hereinbefore defined.

When Q¹ is present, preferably Q¹ is halogen, hydroxy, C₁₋₆alkyl or C₁₋₆alkoxy. More preferably, Q¹ is fluorine, chlorine, bromine, hydroxy, C₁₋₄alkyl or C₁₋₄alkoxy. Most preferably, Q¹ is fluorine, chlorine, hydroxy, methyl or methoxy. Especially, Q¹ is fluorine.

When Q¹ is present and Ar is phenyl, preferably Q¹ is at the 4-position of the phenyl ring.

In another embodiment, R¹ is hydrogen or C₁₋₆alkyl. Preferably, R¹ is hydrogen or C₁₋₄alkyl. More preferably, R¹ is hydrogen, methyl or ethyl. Especially, R¹ is methyl.

In another embodiment, R² is hydrogen or C₁₋₄alkyl. Preferably, R² is hydrogen, methyl or ethyl. Especially, R² is hydrogen.

In another embodiment, R³ is hydrogen, halo, NR^{a}R^{b}, Het or heteroaryl, optionally substituted by C₁₋₄alkyl or C(O)C₁₋₄alkyl, where R^{a} and R^{b} are as hereinbefore defined. Preferably, R³ is hydrogen, fluoro, chloro, bromo, NR^{a}R^{b}, Het or heteroaryl, optionally substituted by methyl or C(O)CH₃, where R^{a} and R^{b} are as hereinbefore defined. Examples of suitable R³ groups include: hydrogen, bromo, NH₂, N(CH₃)₂, NH—SO₂CH₃, N(CH₃)—SO₂CH₃,

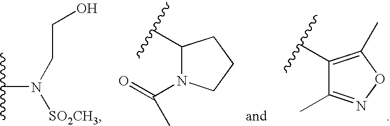

In another embodiment, R⁴ is hydrogen, hydroxy, NR^{c}R^{d}, heteroaryl, O-heteroaryl, C(O)OC₁₋₄alkyl or C(O)NR^{c}R^{d}, optionally substituted by C₁₋₄alkyl or oxo, where R^{c} and R^{d} are as hereinbefore defined. Preferably, R⁴ is hydrogen, hydroxy, NR^{c}R^{d}, heteroaryl, O-heteroaryl, C(O)C₁₋₂alkyl or C(O)NR^{c}R^{d}, optionally substituted by oxo, where R^{c} and R^{d} are independently selected from C₁₋₄alkyl or phenyl, or where R^{c} and R^{d}, together with the nitrogen atom to which they are attached, form a 6-membered heteroaliphatic ring optionally containing one O atom and/or one NH or NC₁₋₄alkyl group. Examples of suitable R⁴ groups include hydrogen, hydroxy, C(O)OCH₃, C(O)N(CH₃)₂, C(O)N(CH₃)phenyl,

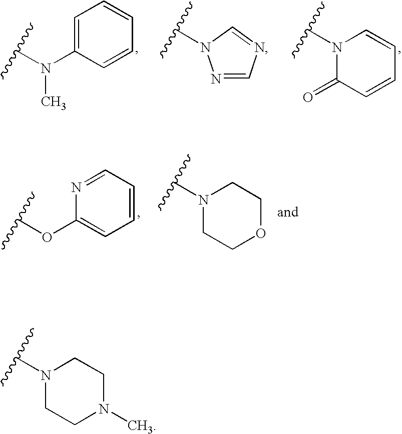

In another embodiment, R⁵ is hydrogen or C₁₋₆alkyl. Preferably, R⁵ is hydrogen or C₁₋₄alkyl. More preferably, R⁵ is hydrogen or C₁₋₂alkyl. Especially, R⁵ is hydrogen or methyl.

One favoured group of compounds of the present invention is the compound of formula (Ia) and pharmaceutically acceptable salts thereof:

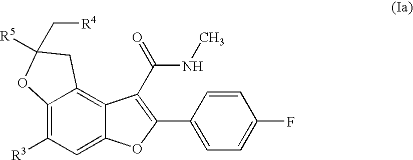

where R³, R⁴ and R⁵ are as defined in relation to formula (I).

When any variable occurs more than one time in formula (I) or in any substituent, its definition on each occurrence is independent of its definition at every other occurrence.

As used herein, the term “alkyl” or “alkoxy” as a group or part of a group means that the group is straight or branched. Examples of suitable alkyl groups include methyl, ethyl, n-propyl, i-propyl, n-butyl, s-butyl and t-butyl. Examples of suitable alkoxy groups include methoxy, ethoxy, n-propoxy, i-propoxy, n-butoxy, s-butoxy and t-butoxy.

The cycloalkyl groups referred to herein may represent, for example, cyclopropyl, cyclobutyl, cyclopentyl or cyclohexyl. A suitable cycloalkylalkyl group may be, for example, cyclopropylmethyl.

As used herein, the term “alkenyl” as a group or part of a group means that the group is straight or branched. Examples of suitable alkenyl groups include vinyl and allyl.

When used herein, the term “halogen” means fluorine, chlorine, bromine and iodine.

When used herein, the term “aryl” as a group or part of a group means a carbocyclic aromatic ring. Examples of suitable aryl groups include phenyl and naphthyl.

When used herein, the term “heteroaryl” as a group or part of a group means a 5- to 10-membered heteroaromatic ring system containing 1 to 4 heteroatoms selected from N, O and S. Particular examples of such groups include pyrrolyl, furanyl, thienyl, pyridyl, pyrazolyl, imidazolyl, oxazolyl, isoxazolyl, thiazolyl, isothiazolyl, pyrazinyl, pyrimidinyl, pyridazinyl, triazolyl, oxadiazolyl, thiadiazolyl, triazinyl, tetrazolyl, indolyl, benzothienyl, benzimidazolyl and quinolinyl.

When used herein, the term “Het” as a group or part of a group means a heteroaliphatic ring of 4 to 7 atoms, which ring may contain 1, 2 or 3 heteroatoms selected from N, O and S or a group S(O), S(O)₂, NH or NC₁₋₄alkyl.

Where a compound or group is described as “optionally substituted” one or more substituents may be present. Optional substituents may be attached to the compounds or groups which they substitute in a variety of ways, either directly or through a connecting group of which the following are examples: amine, amide, ester, ether, thioether, sulfonamide, sulfamide, sulfoxide, urea, thiourea and urethane. As appropriate an optional substituent may itself be substituted by another substituent, the latter being connected directly to the former or through a connecting group such as those exemplified above.

Specific compounds within the scope of this invention include those named in the Examples and Tables below and their pharmaceutically acceptable salts.

For use in medicine, the salts of the compounds of formula (I) will be non-toxic pharmaceutically acceptable salts. Other salts may, however, be useful in the preparation of the compounds according to the invention or of their non-toxic pharmaceutically acceptable salts. Suitable pharmaceutically acceptable salts of the compounds of this invention include acid addition salts which may, for example, be formed by mixing a solution of the compound according to the invention with a solution of a pharmaceutically acceptable acid such as hydrochloric acid, fumaric acid, p-toluenesulfonic acid, maleic acid, succinic acid, acetic acid, citric acid, tartaric acid, carbonic acid, phosphoric acid or sulfuric acid. Salts of amine groups may also comprise quaternary ammonium salts in which the amino nitrogen atom carries a suitable organic group such as an alkyl, alkenyl, alkynyl or aralkyl moiety. Furthermore, where the compounds of the invention carry an acidic moiety, suitable pharmaceutically acceptable salts thereof may include metal salts such as alkali metal salts, e.g. sodium or potassium salts; and alkaline earth metal salts, e.g. calcium or magnesium salts.

The salts may be formed by conventional means, such as by reacting the free base form of the product with one or more equivalents of the appropriate acid in a solvent or medium in which the salt is insoluble, or in a solvent such as water which is removed in vacuo or by freeze drying or by exchanging the anions of an existing salt for another anion on a suitable ion exchange resin.

The present invention includes within its scope prodrugs of the compounds of formula (I) above. In general, such prodrugs will be functional derivatives of the compounds of formula (I) which are readily convertible in vivo into the required compound of formula (I). Conventional procedures for the selection and preparation of suitable prodrug derivatives are described, for example, in “Design of Prodrugs”, ed. H. Bundgaard, Elsevier, 1985.

A prodrug may be a pharmacologically inactive derivative of a biologically active substance (the “parent drug” or “parent molecule”) that requires transformation within the body in order to release the active drug, and that has improved delivery properties over the parent drug molecule. The transformation in vivo may be, for example, as the result of some metabolic process, such as chemical or enzymatic hydrolysis of a carboxylic, phosphoric or sulfate ester, or reduction or oxidation of a susceptible functionality.

The present invention includes within its scope solvates of the compounds of formula (I) and salts thereof, for example, hydrates.

The present invention also includes within its scope N-oxides of the compounds of formula (I).

The present invention also includes within its scope any enantiomers, diastereomers, geometric isomers and tautomers of the compounds of formula (I). It is to be understood that all such isomers and mixtures thereof are encompassed within the scope of the invention.

The present invention further provides a compound of formula (I) or a pharmaceutically acceptable salt thereof for use in therapy.

In another aspect, the invention provides the use of a compound of formula (I) as defined above, or a pharmaceutically acceptable salt thereof, for the manufacture of a medicament for treatment or prevention of infection by hepatitis C virus in a human or animal

A further aspect of the invention provides a pharmaceutical composition comprising a compound of formula (I) as defined above, or a pharmaceutically acceptable salt thereof, in association with a pharmaceutically acceptable carrier. The composition may be in any suitable form, depending on the intended method of administration. It may for example be in the form of a tablet, capsule or liquid for oral administration, or of a solution or suspension for administration parenterally.

The pharmaceutical compositions optionally also include one or more other agents for the treatment of viral infections such as an antiviral agent, or an immunomodulatory agent such as α-, β- or γ-interferon.

In a further aspect, the invention provides a method of inhibiting hepatitis C virus polymerase and/or of treating or preventing an illness due to hepatitis C virus, the method involving administering to a human or animal (preferably mammalian) subject suffering from the condition a therapeutically or prophylactically effective amount of the pharmaceutical composition described above or of a compound of formula (I) as defined above, or a pharmaceutically acceptable salt thereof. “Effective amount” means an amount sufficient to cause a benefit to the subject or at least to cause a change in the subject's condition.

The dosage rate at which the compound is administered will depend on a variety of factors including the activity of the specific compound employed, the metabolic stability and length of action of that compound, the age of the patient, body weight, general health, sex, diet, mode and time of administration, rate of excretion, drug combination, the severity of the particular condition and the host undergoing therapy. Suitable dosage levels may be of the order of 0.02 to 5 or 10 g per day, with oral dosages two to five times higher. For instance, administration of from 10 to 50 mg of the compound per kg of body weight from one to three times per day may be in order. Appropriate values are selectable by routine testing. The compound may be administered alone or in combination with other treatments, either simultaneously or sequentially. For instance, it may be administered in combination with effective amounts of antiviral agents, immunomodulators, anti-infectives or vaccines known to those of ordinary skill in the art. It may be administered by any suitable route, including orally, intravenously, cutaneously and subcutaneously. It may be administered directly to a suitable site or in a manner in which it targets a particular site, such as a certain type of cell. Suitable targeting methods are already known.

An additional aspect of the invention provides a method of preparation of a pharmaceutical composition, involving admixing at least one compound of formula (I) as defined above, or a pharmaceutically acceptable salt thereof, with one or more pharmaceutically acceptable adjuvants, diluents or carriers and/or with one or more other therapeutically or prophylactically active agents.

The present invention also provides a process for the preparation of compounds of formula (I).

According to a general process (a), compounds of formula (I) may be prepared by the conversion of the ester compound of formula (II):

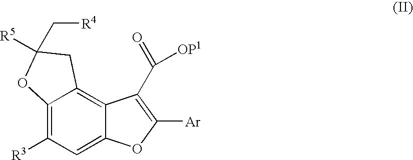

where Ar, R³, R⁴ and R⁵ are as defined in relation to formula (I) and P¹ is a simple alkyl group, to the amide compound of formula (I). This conversion may be carried out in the presence of a suitable amine compound (e.g. HNR¹R², where R¹ and R² are as defined in relation to formula (I)) and a mild base (e.g. PyBOP) in a suitable solvent, such as THF or DMF.

The compound of formula (II) is either known in the art or may be prepared by conventional methodology well known to one of ordinary skill in the art using, for instance, procedures which will be readily apparent.

For example, the compounds of formula (II) may be prepared by internal ring closure of the compound of formula (III):

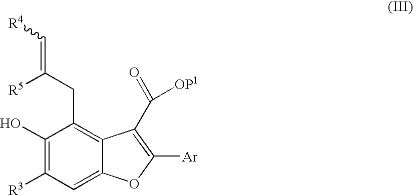

where Ar, R³, R⁴ and R⁵ are as defined in relation to formula (I) and P¹ is a suitable alkyl group as in the compound of formula (II). The intramolecular ring closure may be carried out in the presence of an acid, such as p-TSA or m-CPBA, in a suitable solvent, such as toluene, DCM or NMP, at ambient or raised temperature.

The compound of formula (III) is either known in the art or may be prepared by conventional methodology well known to one of ordinary skill in the art using, for instance, procedures which will be readily apparent.

### General Synthetic Schemes

To date, one principal strategy has been employed for assembly of compounds from the dihydrofuranobenzofuran class (Method A).

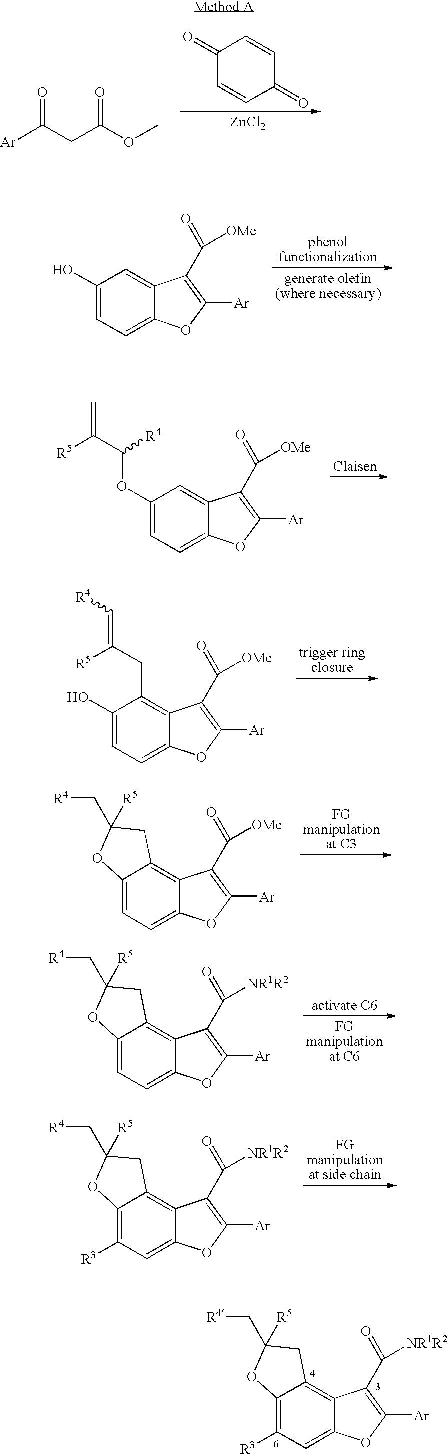

Following 5-hydroxybenzofuran assembly, the phenol is substituted either with a group bearing overtly an allylic double bond or a suitable precursor moiety from which the double bond can be generated. Once the olefin is unmasked, a Claisen rearrangement transfers the functionality to the C4 position of the benzofuran in regioselective fashion. Activation of the newly created olefinic bond (e.g., via H⁺, oxidative) or phenol (e.g., base)—depending on the substituents—promotes ring closure to set up the dihydrofuranobenzofuran core. Functional group manipulation at C3, possibly followed by C6 activation and functional group manipulation and, where appropriate, functional group manipulation in the dihydrofuran side chain affords the target molecules. Where opportune, it is possible to change the order of, or delete, the elaboration steps around the core scaffold.

During any of the above synthetic sequences it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 3rd edition, 1999. The protecting groups may be removed at a convenient subsequent stage using methods known in the art.

The intermediates shown above are either known in the art or may be prepared by conventional methodology well known to one of ordinary skill in the art using, for instance, procedures described in the accompanying Examples, or by alternative procedures which will be readily apparent.

The skilled addressee will appreciate that compounds of formula (I) can be converted into other compounds of formula (I) using synthetic methodology well known in the art. For instance, the compound of formula (I) where R³ is —NHS(O)₂CH₃ may be converted into the compound of formula (I) where R³ is —N(CH₃)S(O)₂CH₃ by alkylation using methyl iodide in the presence of a base, such as K₂CO₃, in a suitable solvent, such as DMF.

In another example, the compound of formula (I) where R³ is hydrogen may be converted into the compound of formula (I) where R³ is NH₂ by nitrosylation using nitric acid in a suitable solvent, such as CHCl₃, followed by hydrogenation using hydrogen in the presence of a suitable catalyst, such as palladium on carbon, in a suitable solvent such as EtOAc. The compound of formula (I) where R³ is NH₂ may itself be further transformed into the compound of formula (I) where R³ is —NHS(O)₂CH₃ by using methanesulfonyl chloride in the presence of a mild base, such as DIPEA or Et₃N, in a suitable solvent, such as DCM.

The compound of formula (I) where R³ is NH₂ may also be transformed into the compound of formula (I) where R³ is N(CH₃)₂ by using formaldehyde in acidic conditions in the presence of a reducing agent, such as sodium cyanoborohydride, in a suitable solvent, such as methanol.

The compound of formula (I) where R³ is hydrogen may also be converted into the compound of formula (I) where R³ is bromine by bromination using bromine in a suitable medium, such as acetic acid.

The compound of formula (I) where R³ is bromine may itself be transformed into a variety of compounds of formula (I) where R³ is heteroaryl by Suzuki coupling methods using a palladium catalyst, such as Pd(PPh₃)₄ and a suitable heteroaryl boronic acid, such as 3,5-dimethylisoxazol-4-ylboronic acid or 1-(tert-butoxycarbonyl)-1H-pyrrol-2-ylboronic acid, in the presence of a buffer, such as Na₂CO₃, in a suitable solvent, such as a water/toluene/ethanol mixture.

Furthermore, the compound of formula (I) where R⁴ is hydroxy may be converted into the compound of formula (I) where R⁴ is NR^{c}R^{d} by activation of the hydroxy group using methanesulfonyl chloride in the presence of a mild base, such as Et₃N, in a suitable solvent, such as DCM, followed by reaction with HNR^{c}R^{d} in the presence of a base, such as K₂CO₃, in a suitable solvent, such as DMF.

The following Examples are illustrative of this invention.

The compounds of the invention were tested for inhibitory activity against the HCV RNA dependent RNA polymerase (NS5B) in an enzyme inhibition assay (example i)) and in a cell based sub-genomic replication assay (example 11)). The compounds generally have IC50's below 10 μM in the enzyme assay and several examples have EC50's below 5 μM in the cell based assay.

### i) In-vitro HCV NS5B Enzyme Inhibition Assay

WO 96/37619 describes the production of recombinant HCV RdRp from insect cells infected with recombinant baculovirus encoding the enzyme. The purified enzyme was shown to possess in vitro RNA polymerase activity using RNA as template. The reference describes a polymerisation assay using poly(A) and oligo(U) as a primer or an heteropolymeric template. Incorporation of tritiated UTP or NTPs is quantified by measuring acid-insoluble radioactivity. The present inventors have employed this assay to screen the various compounds described above as inhibitors of HCV RdRp.

Incorporation of radioactive UMP was measured as follows. The standard reaction (50 μl) was carried out in a buffer containing 20 mM tris/HCl pH 7.5, 5 mM MgCl₂, 1 mM DTT, 50 mM NaCl, 0.03% N-octylglucoside, 1 μCi [³H]-UTP (40 Ci/mmol, NEN), 10 μM UTP and 10 μg/ml poly(A) or 5 μM NTPs and 5 μg/ml heteropolymeric template. Oligo(U)₁₂ (1 μg/ml, Genset) was added as a primer in the assay working on Poly(A) template. The final NS5B enzyme concentration was 5 nM. The order of assembly was: 1) compound, 2) enzyme, 3) template/primer, 4) NTP. After 1 h incubation at 22° C. the reaction was stopped by adding 50 μl of 20% TCA and applying samples to DE81 filters. The filters were washed thoroughly with 5% TCA containing 1 M Na₂HPO₄/NaH₂PO₄, pH 7.0, rinsed with water and then ethanol, air dried, and the filter-bound radioactivity was measured in the scintillation counter. Carrying out this reaction in the presence of various concentrations of each compound set out above allowed determination of IC₅₀ values by utilising the formula:

% Residual activity=100/(1+[I]IC₅₀)^{S}

where [I] is the inhibitor concentration and “s” is the slope of the inhibition curve.
ii) Cell based HCV Replication Assay

Cell clones that stably maintain subgenomic HCV replicon were obtained by transfecting Huh-7 cells with an RNA replicon identical to I₃₇₇neo/NS3-3′/wt described by Lohmann et al. (1999) (EMBL-genbank No. AJ 242652), followed by selection with neomycin sulfate (G418). Viral replication was monitored by measuring the expression of the NS3 protein by an ELISA assay performed directly on cells grown in 96 wells microtiter plates (Cell-ELISA) using the anti-NS3 monoclonal antibody 10E5/24 (as described in published International application WO02/59321). Cells were seeded into 96 well plates at a density of 10⁴ cells per well in a final volume of 0.1 ml of DMEM/10% FCS. Two hours after plating, 50 μl of DMEM/10% FCS containing a 3× concentration of inhibitor were added, cells were incubated for 96 hours and then fixed for 10′ with ice-cold isopropanol. Each condition was tested in duplicate and average absorbance values were used for calculations. The cells were washed twice with PBS, blocked with 5% non-fat dry milk in PBS+0.1% Triton X100+0.02% SDS (PBSTS) and then incubated o/n at 4° C. with the 10E5/24 mab diluted in Milk/PBSTS. After washing 5 times with PBSTS, the cells were incubated for 3 hours at room temperature with Fc specific anti-mouse IgG conjugated to alkaline phosphatase (Sigma), diluted in Milk/PBSTS. After washing again as above, the reaction was developed with p-Nitrophenyl phosphate disodium substrate (Sigma) and the absorbance at 405/620 nm read at intervals. For calculations, we used data sets where samples incubated without inhibitors had absorbance values comprised between 1 and 1.5. The inhibitor concentration that reduced by 50% the expression of NS3 (IC₅₀) was calculated by fitting the data to the Hill equation,

Fraction inhibition=1−(Ai−b)/(A₀−b)=[I]ⁿ/([I]ⁿ+IC₅₀)

where:
Ai=absorbance value of HBI10 cells supplemented with the indicated inhibitor concentration.
A₀=absorbance value of HBI10 cells incubated without inhibitor.
b=absorbance value of Huh-7 cells plated at the same density in the same microtiter plates and incubated without inhibitor.
n=Hill coefficient.
   iii) General Procedures

All solvents were obtained from commercial sources (Fluka, puriss.) and were used without further purification. With the exception of routine deprotection and coupling steps, reactions were carried out under an atmosphere of nitrogen in oven dried (110° C.) glassware. Organic extracts were dried over sodium sulfate, and were concentrated (after filtration of the drying agent) on rotary evaporators operating under reduced pressure. Flash chromatography was carried out on silica gel following published procedure (W. C. Still et al., J. Org. Chem. 1978, 43, 2923) or on commercial flash chromatography systems (Biotage corporation and Jones Flashmaster II) utilising pre-packed columns.

Reagents were usually obtained directly from commercial suppliers (and used as supplied) but a limited number of compounds from in-house corporate collections were utilised. In the latter case the reagents are readily accessible using routine synthetic steps that are either reported in the scientific literature or are known to those skilled in the art.

¹H NMR spectra were recorded on Bruker AM series spectrometers operating at (reported) frequencies between 300 and 600 MHz. Chemical shifts (δ) for signals corresponding to non-exchangeable protons (and exchangeable protons where visible) are recorded in parts per million (ppm) relative to tetramethylsilane and are measured using the residual solvent peak as reference. Signals are tabulated in the order: multiplicity (s, singlet; d, doublet; t, triplet; q, quartet; m, multiplet; b, broad, and combinations thereof); coupling constant(s) in hertz (Hz); number of protons. Mass spectral (MS) data were obtained on a Perkin Elmer API 100, or Waters MicroMass ZQ, operating in negative (ES⁻) or positive (ES⁺) ionization mode and results are reported as the ratio of mass over charge (m/z) for the parent ion only. Preparative scale HPLC separations were carried out on a Waters Delta Prep 4000 separation module, equipped with a Waters 486 absorption detector or on an automated Waters Fraction Lynx or Gilson preparative system. In all cases compounds were eluted with linear gradients of water and MeCN both containing 0.1% TFA using flow rates between 15 and 40 mL/min.

The following abbreviations are used in the examples, the schemes and the tables: Ac: acetyl; aq.: aqueous; Ar: aryl; atm: atmosphere; cat.: catalytic; dioxan(e): 1,4-dioxane; dppf: (1,1′-bisdiphenylphosphino)ferrocene; 1,2-DCE: 1,2-dichloroethane; DCM: dichloromethane; DIPEA: diisopropylethylamine; DMAP: N,N-dimethylpyridin-4-amine; DME: dimethoxyethane; DMF: dimethylformamide; DMS: dimethylsulfide; DMSO: dimethylsulfoxide; DMP: Dess-Martin Periodinane; EDAC.HCl: 1-ethyl-(3-dimethylaminopropyl)carbodiimide HCl salt; eq.: equivalent(s); Et₃N: triethylamine; EtOAc: ethyl acetate; Et₂O: diethyl ether; EtOH: ethanol; h: hour(s); Et₃SiH: triethylsilane; FC: Flash Chromatography; HOAc: acetic acid; HATU: O-(7-azabenzotriazol-1-yl)-N,N,N′,N′-tetramethyluronium hexafluorophophate; m-CPBA: metachloroperbenzoic acid; Me: methyl; MeCN: acetonitrile; MeOH: methanol; min: minutes; MS: mass spectrum; Ms: methanesulfonyl; NBS: N-bromo succinimide; NMP: N-methylpyrrolidone; PE: petroleum ether; Ph: phenyl; p-TSA: para-toluenesulfonic acid; PyBOP: benzotriazol-1-yloxytris(pyrrolidino)phosphonium hexafluorophosphate; quant.: quantitative; RP-HPLC: reversed phase high-pressure liquid chromatography; RT: room temperature; sat.: saturated; sec: second(s); SFC: Super-critical fluid chromatography; s.s.: saturated solution; TBAF: tetrabutyl ammonium fluoride; TBTU: O-benzotriazol-1-yl-N,N,N′,N′-tetramethyluronium tetrafluoroborate; TFA: trifluoroacetic acid; THF: tetrahydrofuran; THP: tetrahydropyranyl; TMS: trimethylsilyl.

### EXAMPLE 1

### 2-(4-Fluorophenyl)-N,7-dimethyl-7,8-dihydrofurano[3,2-e][1]benzofuran-1-carboxamide

### Step 1: Methyl 2-(4-fluorophenyl)-5-hydroxy-1-benzofuran-3-carboxylate

Methyl 3-(4-fluorophenyl)-3-oxopropanoate (1 eq) in Et₂O (1.8 M) was added dropwise to a stirred slurry of ZnCl₂ (1 eq) in EtOH (2.9 M) at 90° C. A solution of 1,4-benzoquinone (1 eq) in Et₂O (0.3 M) was then added slowly over 3.5 h and the mixture stirred at 90° C. for a further 6 h before cooling overnight. Water was added and the mixture extracted with DCM (4×). The combined organic fractions were dried (Na₂SO₄), filtered and the solvent was evaporated under reduced pressure. DCM (6 mL/g) was added and the resulting precipitate filtered off to give pure product. The filtrate was concentrated slightly and cooled to −20° C. overnight. The resulting precipitated product was combined with the first crop and dried to afford the title compound as a pale yellow solid (19% overall). MS (ES⁺) m/z 287 (M+H)⁺.

### Step 2: methyl 5-(allyloxy)-2-(4-fluorophenyl)-1-benzofuran-3-carboxylate

Allyl bromide (2 eq) was added to a stirred mixture of methyl 2-(4-fluorophenyl)-5-hydroxy-1-benzofuran-3-carboxylate (1 eq) and K₂CO₃ (2.5 eq) in acetone (0.18 M) and the mixture was stirred at 50° C. for 18 h. The mixture was filtered through celite, washing with EtOAc and the solvent was evaporated under reduced pressure. The residue was purified by column chromatography eluting with 10% EtOAc/PE to afford the title compound as a colourless solid (88%). MS (ES⁺) m/z 327 (M+H)⁺.

### Step 3: methyl 4-allyl-2-(4-fluorophenyl)-5-hydroxy-1-benzofuran-3-carboxylate

Methyl 5-(allyloxy)-2-(4-fluorophenyl)-1-benzofuran-3-carboxylate was dissolved in DMF (0.2 M), placed in a sealed tube and heated in a microwave oven at 210° C. for 1 h. The mixture was cooled, hydrochloric acid (1M) was added and the mixture was extracted with EtOAc. The combined organic fractions were washed with brine, dried (Na₂SO₄), filtered and the solvent was evaporated under reduced pressure. The residue was purified by column chromatography, eluting with 10% EtOAc/PE to afford the title compound as a colourless solid (83%). MS (ES⁻) m/z 325 (M−H)⁻.

### Step 4: methyl 2-(4-fluorophenyl)-7-methyl-7,8-dihydrofurano[3,2-e][1]benzofuran-1-carboxylate

PTSA (1.1 eq) was added to a stirred mixture of methyl 4-allyl-2-(4-fluorophenyl)-5-hydroxy-1-benzofuran-3-carboxylate (1 eq) in toluene (0.05 M) and the mixture was stirred at 80° C. for 3 h. The mixture was cooled, aqueous sodium carbonate (1M) was added and the mixture was extracted with EtOAc. The combined organic fractions were washed with brine, dried (Na₂SO₄), filtered and the solvent was evaporated under reduced pressure. The residue was purified by column chromatography, eluting with 5% EtOAc/PE to afford the title compound as a colourless solid (50%). MS (ES⁺) m/z 327 (M+H)⁺.

### Step 5: 2-(4-Fluorophenyl)-7-methyl-7,8-dihydrofurano[3,2-e][1]benzofuran-1-carboxylic acid

Solid KOH (4 eq) was added to a stirred mixture of methyl 2-(4-fluorophenyl)-7-methyl-7,8-dihydrofurano[3,2-e][1]benzofuran-1-carboylate (1 eq) in THF/MeOH/H₂O (1:3:3, 0.01 M) and the mixture was stirred at 80° C. Hydrochloric acid (1M) was added and the volatiles evaporated leaving solid in an aqueous solution. The mixture was filtered and the solid dried on the pump to afford the title compound as a cream solid (79%). MS (ES⁻) m/z 311 (M−H)⁻.

### Step 6: 2-(4-Fluorophenyl)-N,7-dimethyl-7,8-dihydrofurano[3,2-e][1]benzofuran-1-carboxamide

Methylamine in THF (2M, 26 eq) was added to a mixture of 2-(4-fluorophenyl)-7-methyl-7,8-dihydrofurano[3,2-e][1]benzofuran-1-carboxylic acid (1 eq) and PyBOP (1 eq) and the mixture was stirred at RT overnight. Aqueous ammonium chloride (saturated) was added and the mixture was extracted with EtOAc. The combined organic fractions were washed with brine, dried (Na₂SO₄), filtered and the solvent was evaporated under reduced pressure. The crude was then purified by prep RP-HPLC (stationary phase: column Waters XTERRA prep. C18, 5 um, 19×150 mm. Mobile phase: MeCN/H₂O buffered with 0.1% TFA). Fractions containing the pure compound were combined and freeze dried to afford the title compound as a colourless solid (31%). ¹H NMR (300 MHz, DMSO-d₆, 300 K) δ 1.42 (d, J 6.2, 3H), 2.81 (d, J 4.3, 3H), 2.82-2.90 (m, 1H), 3.36-3.41 (m obscured by H₂O, 1H), 4.95-5.03 (m, 1H), 6.80 (d, J 8.6, 1H), 7.34-7.40 (m, 3H), 7.86-7.91 (m, 2H), 8.51 (m, 1H); MS (ES⁺) m/z 326 (M+H)⁺.

### EXAMPLE 2

### 2-(4-Fluorophenyl)-N,7-dimethyl-7,8-dihydrofurano[3,2-e][1]benzofuran-1-carboxamide

### Step 1: methyl 2-(4-fluorophenyl)-7-methyl-5-nitro-7,8-dihydrofurano[3,2-e][1]benzofuran-1-carboxylate

A solution of methyl 2-(4-fluorophenyl)-7-methyl-7,8-dihydrofurano[3,2-e][1]benzofuran-1-carboxylate (prepared as described in Example 1, Step 4) in CHCl₃ (0.3 M) was slowly added to a flask containing nitric acid (10 eq) in CHCl₃ (20 M) at 0° C. (ice bath). Then the ice bath was removed and the reaction was stirred at RT for 2 h. The mixture was diluted with water, the organic layer was separated and the water layer was extracted with DCM. The combined organic layers were dried (Na₂SO₄), filtered and concentrated in vacuo to afford the crude product which was used in the next step without further purification (95%). MS (ES⁺) m/z 372 (M+H)⁺.

### Step 2: methyl 5-amino-2-(4-fluorophenyl)-7-methyl-7,8-dihydrofurano[3,2-e][1]benzofuran-1-carboxylate

Methyl 2-(4-fluorophenyl)-7-methyl-5-nitro-7,8-dihydrofurano[3,2-e][1]benzofuran-1-carboxylate was dissolved in EtOAc (0.05 M), and 10% Pd/C (0.2 wt eq) was added. The mixture was degassed and then put under H₂ atmosphere (balloon) overnight. The mixture was filtered and concentrated in vacuo to afford the crude product which was used in the next step without further purification (95%). MS (ES⁺) m/z 342 (M+H)⁺.

### Step 3: methyl 2-(4-fluorophenyl)-7-methyl-5-[(methylsulfonyl)amino]-7,8-dihydrofurano[3,2-e][1]benzofuran-1-carboxylate

MsCl (1.03 eq) was added to a solution of methyl 5-amino-2-(4-fluorophenyl)-7-methyl-7,8-dihydrofurano[3,2-e][1]benzo furan-1-carboxylate in anhydrous DCM (0.15 M) and DIPEA (1.09 eq). The mixture was stirred at RT overnight, before being diluted with DCM, washed with 5 N HCl, s.s. sodium bicarbonate and brine, dried (Na₂SO₄), filtered and concentrated in vacuo. The residue was purified by FC to afford the title compound (60%). MS (ES⁺) m/z 420 (M+H)⁺.

### Step 4: 2-(4-fluorophenyl)-7-methyl-5-[(methylsulfonyl)amino]-7,8-dihydrofurano[3,2-e][1]benzofuran-1-carboxylic acid

NaOH (5 eq) was added to a solution of methyl 2-(4-fluorophenyl)-7-methyl-5-[(methylsulfonyl)amino]-7,8-dihydrofurano[3,2-e][1]benzofuran-1-carboxylate in EtOH (0.12 M). The mixture was stirred at 50° C. for 3 h, cooled and evaporated in vacuo. The residue was brought to pH 3 with 5 N HCl and extracted with EtOAc. The organics were washed with brine, dried (Na₂SO₄), filtered and concentrated in vacuo to afford the crude product which was used in the next step without further purification (67%). MS (ES⁺) m/z 406 (M+H)⁺.

### Step 5: 2-(4-fluorophenyl)-N,7-dimethyl-5-[(methylsulfonyl)amino]-7,8-dihydrofurano[3,2-e][1]benzofuran-1-carboxamide

Methylamine (4.6 eq) was added to a solution 2-(4-fluorophenyl)-7-methyl-5-[(methylsulfonyl)amino]-7,8-dihydrofurano[3,2-e][1]benzofuran-1-carboxylic acid in DMF (0.05 M). PyBOP (1.2 eq) was added and the mixture stirred at RT overnight. The mixture was diluted with water and extracted with EtOAc. The organic layer was washed with brine, dried (Na₂SO₄), filtered and concentrated in vacuo. The residue was purified by HPLC to give the title compound (21%). ¹H-NMR (300 MHz, DMSO-d₆, 300 K) δ 9.29 (s, 1H), 8.48-8.52 (m, 1H), 7.83-7.87 (m, 2H), 7.32-7.36 (m, 2H), 7.29 (s, 1H), 4.99-5.08 (m, 1H), 2.85-3.45 (m, 2H), 2.97 (s, 3H), 2.77-2.78 (d, J 4.5, 3H), 1.40-1.41 (d, J 6.1, 3H); MS (ES⁺) m/z 419 (M+H)⁺.

### EXAMPLE 3

### 2-(4-fluorophenyl)-N,7-dimethyl-5-[methyl(methylsulfonyl)amino]-7,8-dihydrofurano[3,2-e][1]benzofuran-1-carboxamide

To a mixture of 2-(4-fluorophenyl)-N,7-dimethyl-5-[(methylsulfonyl)amino]-7,8-dihydrofurano[3,2-e][1]benzofuran-1-carboxamide (prepared as described in Example 2, Step 5) (0.1 M) and K₂CO₃ (2.1 eq) in DMF was added CH₃I (1.15 eq) and the mixture was stirred at RT overnight. The mixture was diluted with water and extracted with EtOAc. The organic layer was washed with brine, dried (Na₂SO₄), filtered and concentrated in vacuo. The residue was purified by HPLC to give the title compound (19%). ¹H-NMR (300 MHz, CDCl₃, 300 K) δ 7.76-7.80 (m, 2H), 7.36 (s, 1H), 7.15-7.21 (m, 2H), 5.65-5.73 (m, 1H), 5.04-5.14 (m, 1H), 3.07-3.68 (m, 2H), 3.33 (s, 3H), 2.96 (s, 3H), 2.95 (s, 3H), 1.49-1.51 (d, J 6.4, 3H); MS (ES⁺) m/z 433 (M+H)⁺.

### EXAMPLE 4

### 2-(4-fluorophenyl)-7-(hydroxymethyl)-N-methyl-5-[methyl(methylsulfonyl)amino]-7,8-dihydrofurano[3,2-e][1]benzofuran-1-carboxamide

### Step 1: methyl 2-(4-fluorophenyl)-7-(hydroxymethyl)-7,8-dihydrofurano[3,2-e][1benzofuran-1-carboxylate

To a solution of methyl 4-allyl-2-(4-fluorophenyl)-5-hydroxy-1-benzofuran-3-carboxylate (Example 1, Step 3) in anhydrous DCM (0.15 M), was added m-CPBA (1.5 eq) in one portion and the mixture stirred at RT overnight. The mixture was diluted with DCM and washed with aq.Na₂CO₃. The organics were washed with brine, dried (Na₂SO₄), filtered and concentrated in vacuo. The residue was purified by FC to afford the title compound (38%). MS (ES⁺) m/z 343 (M+H)⁺.

### Step 2: methyl 7-[(acetyloxy)methyl]-2-(4-fluorophenyl)-7,8-dihydrofurano[3,2-e[1benzofuran-1-carboxylate

AcCl (1.4 eq) was added dropwise to a solution of methyl 2-(4-fluorophenyl)-7-(hydroxymethyl)-7,8-dihydrofurano[3,2-e][1]benzofuran-1-carboxylate (0.18 M) and DMAP (2 eq) in DCM at 0° C. under N₂. The mixture was stirred at RT for 1 h and quenched by addition of water. The organic layer was separated, dried (Na₂SO₄), filtered and concentrated in vacuo to afford the crude product which was used in the next step without further purification (61%). MS (ES⁺) m/z 385 (M+H)⁺.

### Step 3: methyl 7-[(acetyloxy)methyl]-2-(4-fluorophenyl)-5-nitro-7,8-dihydrofurano[3,2-e][1]benzofuran-1-carboxylate

Methyl 7-[(acetyloxy)methyl]-2-(4-fluorophenyl)-7,8-dihydrofurano[3,2-e][1]benzofuran-1-carboxylate (0.45 M) in CHCl₃ was added dropwise to a flask containing a solution of nitric acid (10 eq) in CHCl₃ (15 M) at 0° C. Then the ice bath was removed and the reaction was stirred at RT for 1 h. The mixture was diluted with DCM, washed with water and brine, dried (Na₂SO₄), filtered and concentrated in vacuo to afford the crude product which was used in the next step without further purification (81%). MS (ES⁺) m/z 430 (M+H)⁺.

### Step 4: methyl 7-[(acetyloxy)methyl]-5-amino-2-(4-fluorophenyl)-7,8-dihydrofurano[3,2-e][1]benzofuran-1-carboxylate

10% Pd/C (0.1 wt eq) was added to a solution of methyl 7-[(acetyloxy)methyl]-2-(4-fluorophenyl)-5-nitro-7,8-dihydrofurano[3,2-e][1]benzofuran-1-carboxylate in EtOAc (0.16 M). The mixture was degassed and then put under H₂ atmosphere (balloon) at RT overnight. Pd/C was filtered off and the solvent was concentrated in vacuo to afford the crude product, which was used in the next step without further purification (90%). MS (ES⁺) m/z 400 (M+H)⁺.

### Step 5: methyl 7-[(acetyloxy)methyl]-2-(4-fluorophenyl)-5-[(methylsulfonyl)amino]-7,8-dihydrofurano[3,2-e][1]benzofuran-1-carboxylate

MsCl (1.2 eq) was added to a mixture of methyl 7-[(acetyloxy)methyl]-5-amino-2-(4-fluorophenyl)-7,8-dihydrofurano[3,2-e][1]benzo furan-1-carboxylate (0.5 M) and anhydrous Et₃N (1.8 eq) in anhydrous DCM at 0° C. The mixture was stirred at RT overnight. The mixture was washed with NaHCO₃ and brine, dried (Na₂SO₄), filtered and concentrated in vacuo to afford the crude product which was used in the next step without further purification (67%). MS (ES⁺) m/z 477 (M+H)⁺.

### Step 6: 2-(4-fluorophenyl)-7-(hydroxymethyl)-5-[(methylsulfonyl)amino]-7,8-dihydrofurano[3,2-e][1]benzofuran-1-carboxylic acid

NaOH (6 eq) was added to the solution of methyl 7-[(acetyloxy)methyl]-2-(4-fluorophenyl)-5-[(methylsulfonyl)amino]-7,8-dihydrofurano[3,2-e][1]benzofuran-1-carboxylate (0.07 M) in a mixture of THF:ethanol:water (1:2:2). The mixture was heated at reflux for 2 h. Volatiles were removed in vacuo and the residue was brought to pH 3 by addition of 5 N HCl and extracted with EtOAc. The organic layer was washed with brine and concentrated in vacuo to afford the crude product which was used in the next step without further purification (76%). MS (ES⁺) m/z 421 (M+H)⁺.

Step 7: 2-(4-fluorophenyl)-7-(hydroxymethyl)-N-methyl-5-[(methylsulfonyl)amino]-7,8-dihydrofurano[3,2-e][1]benzofuran-1-carboxamide

Methylamine (25 eq) was added to a solution of 2-(4-fluorophenyl)-7-(hydroxymethyl)-5-[(methylsulfonyl)amino]-7,8-dihydrofurano[3,2-e][1]benzofuran-1-carboxylic acid in DMF (0.2 M). PyBOP (1 eq) was then added portionwise. The mixture was stirred at RT overnight. The mixture was diluted with water and extracted with EtOAc. The organic layer was washed with brine, dried (Na₂SO₄), filtered and concentrated in vacuo to afford the crude product which was used in the next step without further purification (73%). MS (ES⁺) m/z 435 (M+H)⁺.

### Step 8: 2-(4-fluorophenyl)-7-(hydroxymethyl)-N-methyl-5-[methyl(methylsulfonyl)amino]-7,8-dihydrofurano[3,2-e][1]benzofuran-1-carboxamide

To a mixture of 2-(4-fluorophenyl)-7-(hydroxymethyl)-N-methyl-5-[(methylsulfonyl)amino]-7,8-dihydrofurano[3,2-e][1]benzofuran-1-carboxamide (0.17 M) and K₂CO₃ (2.2 eq) in DMF was added CH₃I (1.15 eq). The mixture was stirred at RT overnight. The mixture was diluted with water and extracted with EtOAc. The organic layer was washed with brine, dried (Na₂SO₄), filtered and concentrated in vacuo to afford the crude product which was purified by HPLC (30%). ¹H-NMR (300 MHz, CDCl₃, 294.5 K) δ 7.78-7.80 (m, 2H), 7.30 (s, 1H), 7.16-7.20 (t, J 8.5, 2H), 5.78-5.86 (m, 1H), 5.06-5.08 (m, 1H), 3.70-3.92 (m, 2H), 3.32-3.64 (m, 2H), 3.34 (s, 3H), 3.06 (s, 3H), 2.94-2.96 (d, J 4.5, 3H); MS (ES⁺) m/z 449 (M+H)⁺.

### EXAMPLE 5

### 2-(4-fluorophenyl)-N-methyl-5-[methyl(methylsulfonyl)amino]-7-(morpholin-4-ylmethyl)-7,8-dihydrofurano[3,2-e][1]benzofuran-1-carboxamide

### Step 1: {2-(4-fluorophenyl)-1-[(methylamino)carbonyl]-5-[methyl(methylsulfonyl)amino]-7,8-dihydrofurano[3,2-e][1]benzofuran-7-yl}methyl methanesulfonate

MsCl (1.03 eq) was added to a mixture of 2-(4-fluorophenyl)-7-(hydroxymethyl)-N-methyl-5-[methyl(methylsulfonyl)amino]-7,8-dihydrofurano[3,2-e][1]benzofuran-1-carboxamide (prepared as described in Example 4, Step 8) (0.13 M) and anhydrous Et₃N (2.5 eq) in anhydrous DCM at 0° C. The mixture was stirred at RT overnight. The mixture was washed with brine, dried (Na₂SO₄) and filtered through a plug of silica. The solvent was concentrated in vacuo to afford the crude product which was used in the next step without further purification. MS (ES⁺) m/z 549 (M+Na)⁺.

### Step 2: 2-(4-fluorophenyl)-N-methyl-5-[methyl(methylsulfonyl)amino]-7-(morpholin-4-ylmethyl)-7,8-dihydrofurano[3,2-e][1]benzofuran-1-carboxamide

A mixture of {2-(4-fluorophenyl)-1-[(methylamino)carbonyl]-5-[methyl(methylsulfonyl)amino]-7,8-dihydrofurano[3,2-e][1]benzofuran-7-yl}methyl methanesulfonate, morpholine and K₂CO₃ in DMF was stirred at 80° C. overnight. Water was added and the mixture was extracted with EtOAc. The organic layer was separated and concentrated in vacuo, then the residue was purified by HPLC. ¹H-NMR (300 MHz, CDCl₃, 300 K) δ 7.74-7.79 (m, 2H), 7.35 (s, 1H), 7.15-7.21 (m, 2H), 5.69-5.74 (m, 1H), 5.07-5.16 (m, 1H), 3.70-3.73 (m, 4H), 3.22-3.65 (m, 2H), 3.31 (s, 3H), 2.98 (s, 3H), 2.94-2.96 (d, J 4.9, 3H), 2.60-2.80 (m, 2H), 2.57-2.58 (m, 4H); MS (ES⁺) m/z 518 (M+H)⁺.

### EXAMPLE 6

### 5-amino-2-(4-fluorophenyl)-N,7-dimethyl-7,8-dihydrofurano[3,2-e][1]benzofuran-1-carboxamide

### Step 1: 2-(4-fluorophenyl)-N,7-dimethyl-5-nitro-7,8-dihydrofurano[3,2-e][1]benzofuran-1-carboxamide

A solution of 2-(4-fluorophenyl)-N,7-dimethyl-7,8-dihydrofurano[3,2-e][1]benzofuran-1-carboxamide (Example 1, Step 6) (0.15 M) in CHCl₃ was slowly added to a solution of nitric acid (10 eq) in CHCl₃ (1 M) at 0° C. (ice bath). Then the ice bath was removed and the reaction was warmed to RT for 4 h. The organic layer was separated and washed with water. The combined aqueous layers were extracted with DCM and the combined organics were dried (Na₂SO₄), filtered and concentrated in vacuo. The residue was purified by FC to afford the title compound (89%). MS (ES⁺) m/z 371 (M+H)⁺.

### Step 2: 5-amino-2-(4-fluorophenyl)-N,7-dimethyl-7,8-dihydrofurano[3,2-e][1]benzofuran-1-carboxamide

2-(4-fluorophenyl)-N,7-dimethyl-5-nitro-7,8-dihydrofurano[3,2-e][1]benzofuran-1-carboxamide in EtOAc (0.03 M) was treated with 1.5 wt eq of 10% Pd/C and the reaction mixture was stirred at RT under H₂ atmosphere (balloon) overnight. The mixture was filtered and concentrated in vacuo and the residue was purified by preparative HPLC to afford the title compound (65%). ¹H-NMR (300 MHz, CDCl₃, 300 K) δ: 7.73-7.77 (m, 2H), 7.10-7.16 (m, 2H), 6.69 (s, 1H), 5.73 (s, 1H), 4.98-5.07 (m, 1H), 3.53-3.60 (m, 1H), 3.03-3.09 (m, 1H), 2.95 (d, J 5.0, 3H), 1.51 (d, J 6.1, 3H); MS (ES⁺) m/z 341 (M+H)⁺.

### EXAMPLE 7

### 5-(dimethylamino)-2-(4-fluorophenyl)-N,7-dimethyl-7,8-dihydrofurano[3,2-e][1]benzofuran-1-carboxamide

### Step 1: 5-(dimethylamino)-2-(4-fluorophenyl)-N,7-dimethyl-7,8-dihydrofurano[3,2-e][1]benzofuran-1-carboxamide

A mixture of 5-amino-2-(4-fluorophenyl)-N,7-dimethyl-7,8-dihydrofurano[3,2-e][1]benzofuran-1-carboxamide (example 6, step 2) (0.06 M) and 40% aq formaldehyde (3.3 eq) in MeOH was acidified with conc. HCl to give a clear solution. Sodium cyanoborohydride (1.5 eq) was added and the reaction mixture was stirred at RT overnight. The mixture was diluted with water, pH adjusted to 10 and extracted with EtOAc. The organic layer was separated and concentrated in vacuo and the residue was purified by preparative HPLC to afford the title compound (18%). ¹H-NMR (300 MHz, CDCl₃, 300 K) δ 7.73-7.78 (m, 2H), 7.10-7.17 (m, 2H), 6.86 (brs, 1H), 5.76 (brs, 1H), 5.01-5.08 (m, 1H), 3.50-3.59 (m, 1H), 3.01-3.08 (m, 1H), 2.96 (d, J 4.9, 3H), 2.88 (brs, 6H), 1.54 (d, J 6.1, 3H); MS (ES⁺) m/z 369 (M+H)⁺.

### EXAMPLE 8

### 5-bromo-2-(4-fluorophenyl)-N,7-dimethyl-7,8-dihydrofurano[3,2-e][1]benzofuran-1-carboxamide

Bromine (1 eq) was added to the solution of 2-(4-fluorophenyl)-N,7-dimethyl-7,8-dihydrofurano[3,2-e][1]benzofuran-1-carboxamide (Example 1, Step 6) (0.05 M) in acetic acid and the mixture was stirred at RT overnight. 1M Na₂S₂O₃ was added and the mixture was extracted with EtOAc. The organic layer was separated and washed with brine, dried (Na₂SO₄), filtered and concentrated in vacuo. The residue was purified by FC to afford the title compound (70%). ¹H-NMR (300 MHz, CDCl₃, 300 K) δ 7.75-7.80 (m, 2H), 7.45 (s, 1H), 7.14-7.20 (m, 2H), 5.69-5.74 (m, 1H), 5.05-5.13 (m, 1H), 3.11-3.71 (m, 1H), 2.94-2.96 (d, J 4.9, 3H), 1.54-1.56 (m, 3H); MS (ES⁺) m/z 404 (M+H)⁺, 406 (M+H)⁺.

### EXAMPLE 9

### 5-(3,5-dimethylisoxazol-4-yl)-2-(4-fluorophenyl)-N,7-dimethyl-7,8-dihydrofurano[3,2-e][1]benzofuran-1-carboxamide

Pd(PPh₃)₄ (0.09 eq) was added to a solution of 5-bromo-2-(4-fluorophenyl)-N,7-dimethyl-7,8-dihydrofurano[3,2-e][1]benzofuran-1-carboxamide (Example 8) (0.06 M), 3,5-dimethylisoxazol-4-ylboronic acid (1.8 eq) and Na₂CO₃ (4 eq) in water:toluene:ethanol (2:1:1) under argon. The mixture was heated at reflux overnight, before being allowed to cool, filtered and concentrated in vacuo. The residue was purified by FC and then by preparative HPLC to afford the title compound (8%). ¹H-NMR (300 MHz, CDCl₃, 300 K) δ 7.78-7.83 (m, 2H), 7.15-7.21 (m, 2H), 7.10 (s, 1H), 5.83-5.88 (m, 1H), 4.98-5.05 (m, 1H), 3.07-3.66 (m, 1H), 2.98-2.99 (d, J 4.9, 3H), 2.37 (s, 3H), 2.25 (s, 3H), 1.48-1.50 (d, J 6.4, 3H); MS (ES⁺) m/z 421 (M+H)⁺.

### EXAMPLE 10

### 5-(1-acetylpyrrolidin-2-yl)-2-(4-fluorophenyl)-N,7-dimethyl-7,8-dihydrofurano[3,2-e][1]benzofuran-1-carboxamide

### Step 1: tert-butyl 2-{2-(4-fluorophenyl)-7-methyl-1-[(methylamino)carbonyl]-7,8-dihydrofurano[3,2-e][1]benzofuran-5-yl}-1H-pyrrole-1-carboxylate

Pd(PPh₃)₄ (0.1 eq) was added to a mixture of 5-bromo-2-(4-fluorophenyl)-N,7-dimethyl-7,8-dihydrofurano[3,2-e][1]benzofuran-1-carboxamide (Example 8) (0.07 M), 1-(tert-butoxycarbonyl)-1H-pyrrol-2-ylboronic acid (1.08 eq) and Na₂CO₃ (3.1 eq) in water:toluene:ethanol (2:1:1) under N₂. The mixture was heated at reflux overnight before being cooled, concentrated in vacuo and the residue purified by HPLC to afford the title compound (44%). MS (ES⁺) m/z 491 (M+H)⁺.

### Step 2: tert-butyl 2-{2-(4-fluorophenyl)-7-methyl-1-[(methylamino)carbonyl]-7,8-dihydrofurano[3,2-e][1]benzofuran-5-yl}pyrrolidine-1-carboxylate

10% Pd/C (1 wt eq) was added to tert-butyl 2-{2-(4-fluorophenyl)-7-methyl-1-[(methylamino)carbonyl]-7,8-dihydrofurano[3,2-e][1]benzofuran-5-yl}-1H-pyrrole-1-carboxylate (0.03 M) in EtOAc. The mixture was hydrogenated at 45 psi H₂ pressure at 50° C. overnight. The mixture was allowed to cool, filtered and concentrated in vacuo and the residue purified by preparative HPLC to afford the title compound (67%). MS (ES⁺) m/z 495 (M+H)⁺.

### Step 3: 2-(4-fluorophenyl)-N,7-dimethyl-5-pyrrolidin-2-yl-7,8-dihydrofurano[3,2-e][1]benzofuran-1-carboxamide

A large excess of TFA (225 eq) was added to a solution of tert-butyl 2-{2-(4-fluorophenyl)-7-methyl-1-[(methylamino)carbonyl]-7,8-dihydrofurano[3,2-e][1]benzo furan-5-yl}pyrrolidine-1-carboxylate (0.015 M) in DCM and the resulting mixture was stirred at RT for 2 h. The mixture was diluted with EtOAc, washed with aq Na₂CO₃ and brine, dried (Na₂SO₄), filtered and concentrated in vacuo to afford the title compound, which was used in the next step without further purification. MS (ES⁺) m/z 395 (M+H)⁺.

### Step 4: 5-(1-acetylpyrrolidin-2-yl)-2-(4-fluorophenyl)-N,7-dimethyl-7,8-dihydrofurano[3,2-e][1]benzofuran-1-carboxamide

AcCl (1.1 eq) was added dropwise to a solution of 2-(4-fluorophenyl)-N,7-dimethyl-5-pyrrolidin-2-yl-7,8-dihydrofurano[3,2-e][1]benzofuran-1-carboxamide (0.02 M) and pyridine (6.2 eq) in anhydrous DCM at 0° C. under N₂. The resulting mixture was stirred at RT overnight, quenched by addition of water and extracted with EtOAc. The organic layer was separated and washed with brine, dried (Na₂SO₄), filtered and concentrated in vacuo. The residue was purified by preparative HPLC to afford the title compound (38%). ¹H-NMR (300 MHz, CDCl₃, 300 K) δ 7.75-7.80 (m, 2H), 7.14-7.20 (m, 2H), 6.89-6.96 (m, 1H), 5.82-5.88 (m, 1H), 5.12-5.15 (m, 1H), 5.00-5.09 (m, 1H), 3.63-3.85 (m, 2H), 3.01-3.61 (m, 2H), 2.95-2.97 (d, J 4.9, 3H), 2.01-2.43 (m, 2H), 1.97-1.99 (m, 2H), 1.91-1.92 (d, J 3.4, 3H), 1.47-1.50 (m, 3H); MS (ES⁺) m/z 437 (M+H)⁺.

### EXAMPLE 11

### 2-(4-fluorophenyl)-N,7,7-trimethyl-7,8-dihydrofurano[3,2-e][1]benzofuran-1-carboxamide

### Step 1: methyl 2-(4-fluorophenyl)-5-[(2-methylprop-2-en-1-yl)oxy]-1-benzofuran-3-carboxylate

3-Bromo-2-methylprop-1-ene (1.3 eq) was added to a solution of methyl 2-(4-fluorophenyl)-5-hydroxy-1-benzofuran-3-carboxylate (Example 1, Step 1) (0.28 M) and K₂CO₃ (2.5 eq) in acetone. The resulting mixture was stirred at 50° C. overnight. The mixture was filtered, washing with EtOAc and the combined organic layers were concentrated in vacuo. The residue was purified by FC to afford the title compound (42%). MS (ES⁺) m/z 341 (M+H)⁺.

### Step 2: methyl 2-(4-fluorophenyl)-5-hydroxy-4-(2-methylprop-2-en-1-yl)-1-benzofuran-3-carboxylate

Methyl 2-(4-fluorophenyl)-5-[(2-methylprop-2-en-1-yl)oxy]-1-benzofuran-3-carboxylate in NMP (0.1 M) was heated at 190˜200° C. for 7 h. The mixture was cooled and partitioned between water and EtOAc. The organics were washed with brine, dried (Na₂SO₄), filtered and concentrated in vacuo. The residue was purified by FC to afford the title compound (60%). MS (ES⁻) m/z 339 (M−H)⁻.

### Step 3: methyl 2-(4-fluorophenyl)-7,7-dimethyl-7,8-dihydrofurano[3,2-e][1]benzofuran-1-carboxylate

p-TSA (1 eq) was added to a stirred solution of methyl 2-(4-fluorophenyl)-5-hydroxy-4-(2-methylprop-2-en-1-yl)-1-benzofuran-3-carboxylate (0.07 M) in toluene and the mixture was stirred at 80° C. overnight. The mixture was cooled, diluted with EtOAc, washed with aq Na₂CO₃, water and brine, dried (Na₂SO₄), filtered and concentrated in vacuo. The residue was purified by FC to afford the title compound (60%). MS (ES⁺) m/z 341 (M+H)⁺.

### Step 4: 2-(4-fluorophenyl)-7,7-dimethyl-7,8-dihydrofurano[3,2-e][1]benzofuran-1-carboxylic acid

NaOH (5 eq) was added to a solution of methyl 2-(4-fluorophenyl)-7,7-dimethyl-7,8-dihydrofurano[3,2-e][1]benzofuran-1-carboxylate (0.3 M) in ethanol and the mixture was heated at reflux for 4 h. The mixture was allowed to cool before diluting with EtOAc and water. The water fraction was acidified to pH 3 with 5 N HCl and extracted with EtOAc. The organic layer was separated and washed with brine, dried (Na₂SO₄), filtered and concentrated in vacuo to afford the crude product which was used in the next step without further purification (60%). MS (ES⁺) m/z 326 (M+H)⁺.

### Step 5: 2-(4-fluorophenyl)-N,7,7-trimethyl-7,8-dihydrofurano[3,2-e][1]benzofuran-1-carboxamide

PyBOP (1 eq) was added to a solution of 2-(4-fluorophenyl)-7,7-dimethyl-7,8-dihydrofurano[3,2-e][1]benzofuran-1-carboxylic acid (0.12 M) and methylamine (10 eq) in DMF at 0° C. The resulting mixture was stirred at RT overnight before pouring into water and filtering the precipitate to afford crude product. Purification was by HPLC. ¹H-NMR (300 MHz, CDCl₃, 300 K) δ 7.76-7.80 (m, 2H), 7.23-7.26 (m, 1H), 7.10-7.17 (m, 2H), 6.73-6.75 (m, 1H), 5.83 (s, 1H), 3.34 (s, 3H), 2.89-2.91 (d, J 6.1, 3H), 2.15 (s, 6H); MS (ES⁺) m/z 340 (M+H)⁺.

### EXAMPLE 12

### methyl {2-(4-fluorophenyl)-1-[(methylamino)carbonyl]-7,8-dihydrofurano[3,2-e][1]benzofuran-7-yl}acetate

### Step 1: 2-(4-fluorophenyl)-5-hydroxy-1-benzofuran-3-carboxylic acid

Methyl 2-(4-fluorophenyl)-5-hydroxy-1-benzofuran-3-carboxylate (Example 1, Step 1) (0.3 M) in EtOH:water:THF (4:4:1) was treated with NaOH (4.25 eq) and the mixture heated at reflux for 2 h, before being allowed to cool. The mixture was concentrated in vacuo and the residue acidified with hydrochloric acid and extracted with EtOAc. The organics were washed with water and brine, dried (Na₂SO₄), filtered and concentrated in vacuo to afford crude product which was used in the next step without further purification. MS (ES⁺) m/z 273 (M+H)⁺.

### Step 2: 2-(4-fluorophenyl)-5-hydroxy-N-methyl-1-benzofuran-3-carboxamide

PyBOP (1 eq) was added to a mixture of 2-(4-fluorophenyl)-5-hydroxy-1-benzofuran-3-carboxylic acid (0.2 M) and MeNH₂ (3.9 M in THF, 6.5 eq) in DMF at 0° C. The resulting mixture was stirred at RT overnight, and then partitioned between EtOAc and water. The organic layer was separated and washed with water and brine, dried (Na₂SO₄), filtered and concentrated in vacuo. The crude product was washed with MeCN and used in the next step without further purification. MS (ES⁺) m/z 286 (M+H)⁺.

### Step 3: 2-(4-fluorophenyl)-N-methyl-5-[(2-oxotetrahydrofuran-3-yl)oxy]-1-benzofuran-3-carboxamide

A mixture of 2-(4-fluorophenyl)-5-hydroxy-N-methyl-1-benzofuran-3-carboxamide (0.3 M), 3-bromodihydrofuran-2(3H)-one (1.24 eq) and K₂CO₃ (2.2 eq) in MeCN was heated at 80° C. overnight. The mixture was cooled, filtered and concentrated in vacuo to afford crude product which was use in the next step without further purification. MS (ES⁺) m/z 370 (M+H)⁺.

### Step 4: methyl 2-({2-(4-fluorophenyl)-3-[(methylamino)carbonyl]-1-benzofuran-5-yl}oxy)-4-(phenylseleno)butanoate

A solution of diphenyldiselane (0.55 eq) was treated with NaBH₄ (1.25 eq) under N₂. The temperature of the mixture was raised to 100° C. over 30 min, 2-(4-fluorophenyl)-N-methyl-5-[(2-oxotetrahydrofuran-3-yl)oxy]-1-benzo furan-3-carboxamide (0.6 M) in DMF was added dropwise, and the mixture was heated to 125° C. and stirred at this temperature for 2 h. The mixture was cooled and partitioned between EtOAc and water, and then the organic layer was separated and concentrated in vacuo to afford the carboxylic acid intermediate. The crude acid was dissolved in MeOH (0.12 M) and treated with conc. H₂SO₄ (8 eq). The mixture was stirred at RT for 2 h, partitioned between EtOAc and water. The organic layer was separated, dried (Na₂SO₄), filtered and concentrated in vacuo. Purification was by FC to afford the title compound (46%). MS (ES⁺) m/z 542 (M+H)⁺.

### Step 5: methyl 2-({2-(4-fluorophenyl)-3-[(methylamino)carbonyl]-1-benzofuran-5-yl}oxy)but-3-enoate

A solution of methyl 2-({2-(4-fluorophenyl)-3-[(methylamino)carbonyl]-1-benzofuran-5-yl}oxy)-4-(phenylseleno)butanoate (0.04 M) in THF was treated with H₂O₂ (30 wt % in water; 14 eq) at RT for 3 h. The mixture was partitioned between EtOAc and water. The organic layer was separated and washed with aq Na₂CO₃, water and brine, dried (Na₂SO₄), filtered and concentrated in vacuo. Purification was by FC to afford the title compound (83%). MS (ES⁺) m/z 384 (M+H)⁺.

### Step 6: methyl (2E)-4-{2-(4-fluorophenyl)-5-hydroxy-3-[(methylamino)carbonyl]-1-benzofuran-4-yl}but-2-enoate

Methyl 2-({2-(4-fluorophenyl)-3-[(methylamino)carbonyl]-1-benzofuran-5-yl}oxy)but-3-enoate (0.11 M) in NMP was heated at 170° C. under N₂ for 1 h. The mixture was cooled and partitioned between EtOAc and water. The organic layer was separated and concentrated in vacuo, before purifying by FC to afford the title compound (60%). MS (ES⁺) m/z 384 (M+H)⁺.

### Step 7: methyl {2-(4-fluorophenyl)-1-[(methylamino)carbonyl]-7,8-dihydrofurano[3,2-e][1]benzofuran-7-yl}acetate

A solution of methyl (2E)-4-{2-(4-fluorophenyl)-5-hydroxy-3-[(methylamino)carbonyl]-1-benzofuran-4-yl}but-2-enoate (0.4 M) in MeOH was treated with 10% aq Na₂CO₃ solution (0.05 eq). The mixture was stirred at RT for 3 h and then partitioned between EtOAc and water. The organic layer was separated, dried (Na₂SO₄), filtered and concentrated in vacuo. The crude product was purified by FC to afford the title compound (93%). ¹H-NMR (300 MHz, CDCl₃, 300 K) δ 7.79-7.83 (m, 2H), 7.24-7.26 (m, 1H), 7.15-7.20 (m, 2H), 6.81 (d, J 8.6, 1H), 5.75 (brs, 1H), 5.26-5.31 (m, 1H), 3.75 (s, 3H), 3.63-3.69 (m, 1H), 3.16-3.22 (m, 1H), 2.96 (d, J 5.1, 3H), 2.87-2.93 (m, 1H), 2.70-2.75 (m, 1H); MS (ES⁺) m/z 384 (M+H)⁺.

The following tables list specific compounds of the present invention. The tables provide the structure and name of each compound and the mass of its molecular ion plus 1 (M+1) as determined via ES-MS. All compounds were prepared according to the principles set out in General Synthetic Method A.

TABLE 1

| Example | | | Mass spec |
|---|---|---|---|
| no. | Structure | Compound name | (M + 1) |
| 1/101 | 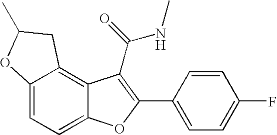 | 2-(4-fluorophenyl)-N,7-dimethyl-7,8- dihydrofurano[3,2-e][1]benzofuran-1- carboxamide | 326 |
| 2/102 | 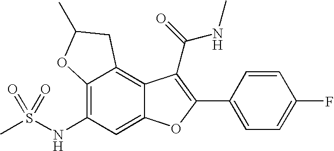 | 2-(4-fluorophenyl)-N,7-dimethyl-5- [(methylsulfonyl)amino]-7.8-dihydrofurano [3,2-e][1]benzofuran-1-carboxamide | 419 |
| 3/103 | 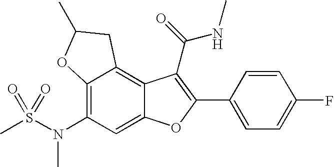 | 2-(4-fluorophenyl)-N,7-dimethyl-5- [methyl(methylsulfonyl)amino]-7,8- dihydrofurano[3,2-e][1]benzofuran-1- carboxamide | 433 |
| 104 | 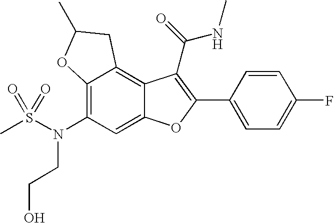 | 2-(4-fluorophenyl)-5-[(2- hydroxyethyl)(methylsulfonyl)amino]-N,7- dimethyl-7,8-dihydrofurano[3,2- e][1]benzofuran-1-carboxamide | 463 |
| 8/105 | 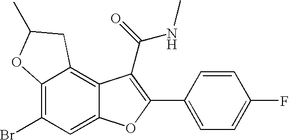 | 5-bromo-2-(4-fluorophenyl)-N,7-dimethyl- 7,8-dihydrofurano[3,2-e][1]benzofuran-1- carboxamide | 404/406 |
| 6/106 | 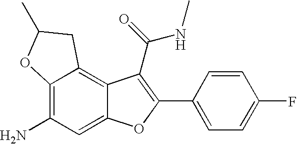 | 5-amino-2-(4-fluroophenyl)-N,7-dimethyl- 7,8-dihydrofurano[3,2-e][1]benzofuran-1- carboxamide | 341 |
| 7/107 | 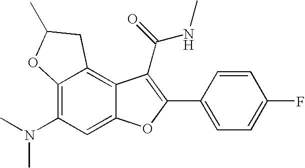 | 5-(dimethylamino)-2-(4-fluorophenyl)-N,7- dimethyl-7,8-dihydrofurano[3,2- e][1]benzofuran-1-carboxamide | 369 |
| 10/108 | 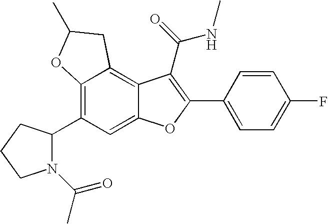 | 5-(1-acetylpyrrolidin-2-yl)-2-(4- fluorophenyl)-N,7-dimethyl-7,8- dihydrofurano[3,2-e][1]benzofuran-1- carboxamide | 437 |
| 9/109 | 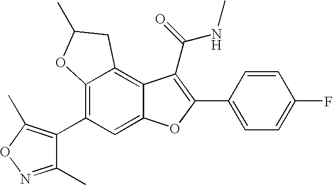 | 5-(3,5-dimethylisoxazol-4-yl)-2-(4- fluorophenyl)-N,7-dimethyl-7,8- dihydrofurano[3,2-e][1]benzofuran-1- carboxamide | 421 |
| 110 | 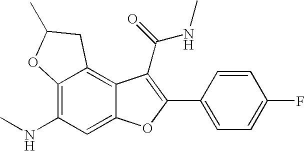 | 5-(methylamino)-2-(4-fluorophenyl)-N,7- dimethyl-7,8-dihydrofurano[3,2- e][1]benzofuran-1-carboxamide | 355 |

TABLE 2

| Example | | | Mass spec |
|---|---|---|---|
| no. | Structure | Compound name | (M + 1) |
| 11/201 | 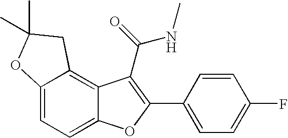 | 2-(4-fluorophenyl)-N,7,7-trimethyl-7,8- dihydrofurano[3,2-e][1]benzofuran-1- carboxamide | 340 |
| 202 | 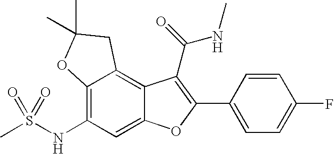 | 2-(4-fluorophenyl)-N,7,7-trimethyl-5- [(methylsulfonyl)amino]-7,8- dihydrofurano[3,2-e][1]benzofuran-1- carboxamide | 433 |
| 203 | 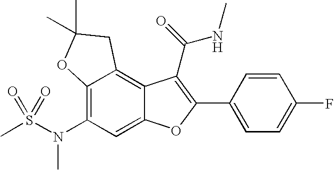 | 2-(4-fluorophenyl)-N,7,7-trimethyl-5- [methyl(methylsulfonyl)amino]-7,8- dihydrofurano[3,2-e][1]benzofuran-1- carboxamide | 447 |
| 4/204 | 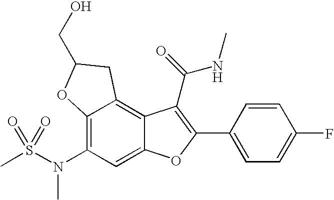 | 2-(4-fluorophenyl)-7-(hydroxymethyl)- N-methyl-5-[methyl(methylsulfonyl) amino]-7,8-dihydrofurano[3,2- e][1]benzofuran-1-carboxamide | 449 |
| 5/205 | 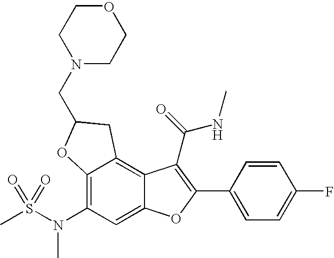 | 2-(4-fluorophenyl)-N-methyl-5- [methyl(methylsulfonyl)amino]-7- (morpholin-4-ylmethyl)-7,8- dihydrofurano[3,2-e][1]benzofuran-1- carboxamide | 518 |
| 206 | 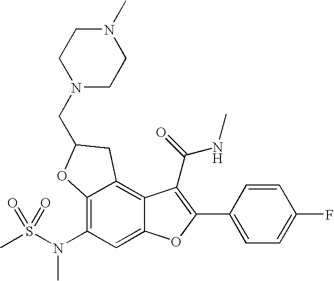 | 2-(4-fluorophenyl)-N-methyl-5- [methyl(methylsulfonyl)maino]-7-[(4- methylpiperazin-1-yl)methyl]-7,8- dihydrofurano[3,2-e][1]benzofuran-1- carboxamide | 531 |
| 207 | 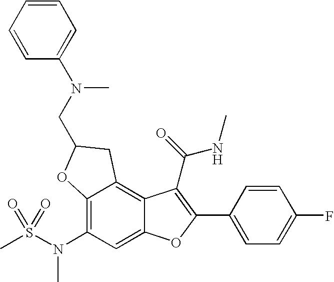 | 2-(4-fluorophenyl)-N-methyl-5- [methyl(methylsulfonyl)amino]-7- {[methyl(phenyl)amino]methyl}-7,8- dihydrofurano[3,2-e][1]benzofuran-1- carboxamide | 538 |
| 208 | 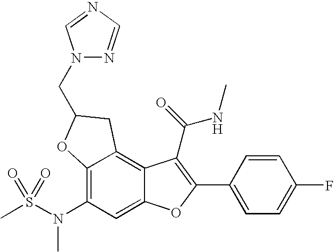 | 2-(4-fluorophenyl)-N-methyl-5- [methyl(methylsulfonyl)amino]-7-(1H- 1,2,4-triazol-1-ylmethyl)-7,8- dihydrofurano[3,2-e][1]benzofuran-1- carboxamide | 500 |
| 209 | 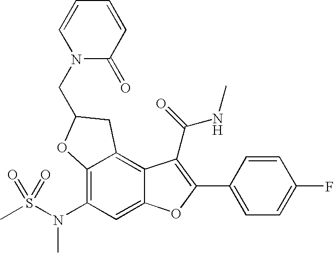 | 2-(4-fluorophenyl)-N-methyl-5- [methyl(methylsulfonyl)amino]-7-[(2- oxopyridin-1(2H)-yl)methyl]-7,8- dihydrofurano[3,2-e][1]benzofuran-1- carboxamide | 526 |
| 210 | 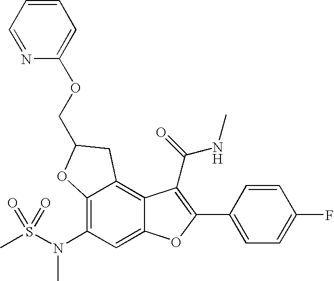 | 2-(4-fluorophenyl)-N-methyl-5- [methyl(methylsulfonyl)amino]-7- [(pyridin-2-yloxy)methyl]-7,8- dihydrofurano[3,2-e][1]benzofuran-1- carboxamide | 526 |
| 12/211 | 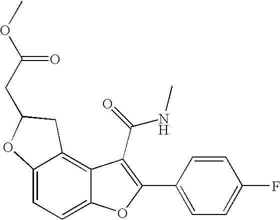 | methyl {2-(4-fluorophenyl)-1- [(methylamino)carbonyl]-7,8- dihydrofurano[3,2-e][1]benzofuran-7- yl}acetate | 384 |
| 212 | 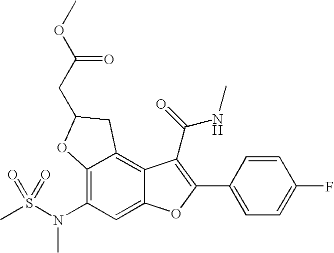 | methyl {2-(4-fluorophenyl)-1- [(methylamino)carbonyl]-5- [methyl(methylsulfonyl)amino]-7,8- dihydrofurano[3,2-e][1]benzofuran-7- yl}acetate | 491 |
| 213 | 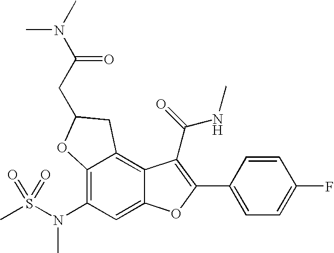 | 7-[2-(dimethylamino)-2-oxoethyl]-2-(4- fluorophenyl)-N-methyl-5- [methyl(methylsulfonyl)amino]-7,8- dihydrofurano[3,2-e][1]benzofuran-1- carboxamide | 504 |
| 214 | 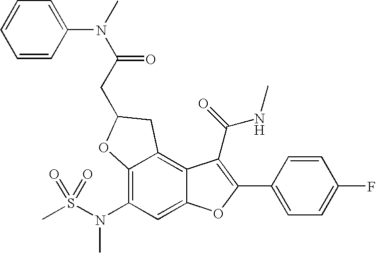 | 2-(4-fluorophenyl)-N-methyl-5- [methyl(methylsulfonyl)amino]-7-{2- [methyl(phenyl)amino]-2-oxoethyl}-7,8- dihydrofurano[3,2-e][1]benzofuran-1- carboxamide | 566 |

1. A compound of the formula (I): 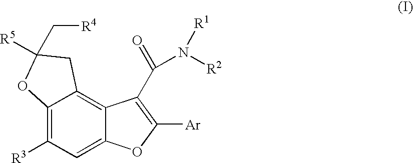
wherein
Ar is a moiety containing at least one aromatic ring and possesses 5, 6, 9 or 10 ring atoms, optionally containing 1, 2 or 3 heteroatoms independently selected from N, O and S, which ring is optionally substituted by groups Q¹ and Q²;
Q¹ is halogen, hydroxy, C₁₋₆alkyl, C₁₋₆alkoxy, (CH₂)₀₋₃aryl, (CH₂)₀₋₃heteroaryl, CONR^{c}R^{d}, (CH₂)₀₋₃NR^{c}R^{d}, O(CH₂)₀₋₃C₃₋₈cycloalkyl, O(CH₂)₁₋₃NR^{c}R^{d}, O(CH₂)₀₋₃CONR^{c}R^{d}, O(CH₂)₀₋₃CO₂H, O(CH₂)₀₋₃aryl, O(CH₂)₀₋₃heteroaryl, OCHR^{e}R^{f} or O(CH₂)₀₋₃S(O)₂(CH₂)₀₋₃NR^{c}R^{d};
R^{c} and R^{d} are independently selected from hydrogen, C₁₋₆alkyl and C(O)C₁₋₆alkyl;
or R^{c} and R^{d}, together with the nitrogen atom to which they are attached, form a heteroaliphatic ring of 4 to 7 ring atoms, optionally containing 1 or 2 more heteroatoms independently selected from O and S and/or 1 or 2 groups independently selected from NH and NC₁₋₄alkyl, where said ring is optionally substituted by halogen, hydroxy, C₁₋₄alkyl or C₁₋₄alkoxy;
R^{e} and R^{f} are independently selected from hydrogen, C₁₋₄alkyl and C₁₋₄alkoxy;
or R^{e} and R^{f} are linked by a heteroatom selected from N, O and S to form a heteroaliphatic ring of 4 to 7 ring atoms, where said ring is optionally substituted by halogen, hydroxy, C₁₋₄alkyl or C₁₋₄alkoxy;
and where said C₁₋₄alkyl, C₁₋₄alkoxy and aryl groups are optionally substituted by halogen or hydroxy;
Q² is halogen, hydroxy, C₁₋₄alkyl or C₁₋₄alkoxy, where said C₁₋₄alkyl and C₁₋₄alkoxy groups are optionally substituted by halogen or hydroxy;
R¹ is hydrogen, C₁₋₆alkyl, C₂₋₆alkenyl, C₂₋₆alkynyl, (CH₂)₀₋₃C₃₋₈cycloalkyl or (CH₂)₀₋₃-phenyl;
R² is hydrogen or C₁₋₆alkyl;
R³ is hydrogen, halogen, hydroxy, C₁₋₆alkyl, C₂₋₆alkenyl, C₂₋₆alkynyl, C₃₋₈cycloalkyl, (CH₂)₀₋₃phenyl, OC₁₋₆alkyl, O(CH₂)₀₋₃C₃₋₈cycloalkyl, O(CH₂)₀₋₃phenyl, NR^{a}R^{b}, Het or heteroaryl, optionally substituted by C₁₋₄alkyl or C(O)C₁₋₄alkyl;
R^{a} and R^{b} are independently selected from hydrogen, C₁₋₆alkyl, C₁₋₆alkylene-OH and SO₂C₁₋₄alkyl;
R⁴ is hydrogen, halo, hydroxy, NR^{c}R^{d}, heteroaryl, O-heteroaryl, C(O)OC₁₋₄alkyl or C(O)NR^{c}R^{d}, optionally substituted by C₁₋₄alkyl, halo, hydroxy or oxo;
R^{c} and R^{d} are independently selected from hydrogen, C₁₋₄alkyl or aryl;
or R^{c} and R^{d}, together with the nitrogen atom to which they are attached, form a 5- or 6-membered heteroaliphatic ring optionally containing 1 or 2 more heteroatoms independently selected from O and S and/or 1 or 2 groups independently selected from S(O), S(O)₂, NH and NC₁₋₄alkyl; and
R⁵ is hydrogen, C₁₋₆alkyl, C₂₋₆alkenyl, C₂₋₆alkynyl or (CH₂)₀₋₃cycloalkyl;
and pharmaceutically acceptable salts thereof.

2. The compound of the formula (I) according to claim 1 in which Ar is phenyl, pyridyl, pyridazinyl, pyrimidinyl, pyrazinyl, thienyl, furanyl, pyrazolyl or imidazolyl, optionally substituted by Q¹.

3. The compound of the formula (I) according to claim 1 in which Q¹ is fluorine, chlorine, bromine, hydroxy, C₁₋₄alkyl or C₁₋₄alkoxy.

4. The compound of the formula (I) according to claim 1 in which R¹ is hydrogen, methyl or ethyl.

5. The compound of the formula (I) according to claim 1 in which R² is hydrogen, methyl or ethyl.

6. The compound of the formula (I) according to claim 1 in which R³ is hydrogen, fluoro, chloro, bromo, NR^{a}R^{b}, Het or heteroaryl, optionally substituted by methyl or C(O)CH₃.

7. The compound of the formula (I) according to claim 1 in which R⁴ is hydrogen, hydroxy, NR^{c}R^{d}, heteroaryl, O-heteroaryl, C(O)C₁₋₂alkyl or C(O)NR^{c}R^{d}, optionally substituted by oxo, where R^{c} and R^{d} are independently selected from C₁₋₄alkyl or phenyl, or where R^{c} and R^{d}, together with the nitrogen atom to which they are attached, form a 6-membered heteroaliphatic ring optionally containing one O atom and/or one NH or NC₁₋₄alkyl group.

8. The compound of the formula (I) according to claim 1 in which R⁵ is hydrogen or C₁₋₂alkyl.

9. The compound of the formula (I) according to claim 1 of formula (Ia) and pharmaceutically acceptable salts thereof:

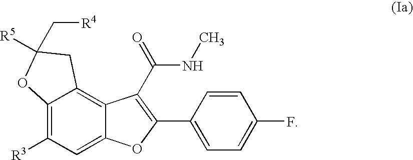

10. A pharmaceutical composition comprising a compound of formula (I) as defined according to claim 1, or a pharmaceutically acceptable salt thereof, in association with a pharmaceutically acceptable carrier.

11. A compound of the formula (I) according to any one of claims 1 to 9 or a pharmaceutically acceptable salts thereof for use in medicine.

12. A method of inhibiting hepatitis C virus polymerase and/or of treating or preventing an illness due to hepatitis C virus, the method involving administering to a human or animal (preferably mammalian) subject suffering from the condition a therapeutically or prophylactically effective amount of the compound of formula (I) according to claim 1, or a pharmaceutically acceptable salt thereof.

13. A method of preparing a medicament for treatment or prevention of infection by hepatitis C virus in a human or animaly, said method comprising providing the compound of formula (I) according to claim 1, or a pharmaceutically acceptable salt thereof.

14. A compound of the formula (I) selected from:
2-(4-fluorophenyl)-N,7-dimethyl-7,8-dihydrofurano[3,2-e][1]benzofuran-1-carboxamide;
2-(4-fluorophenyl)-N,7-dimethyl-5-[(methylsulfonyl)amino]-7,8-dihydrofurano[3,2-e][1]benzofuran-1-carboxamide;
2-(4-fluorophenyl)-N,7-dimethyl-5-[methyl(methylsulfonyl)amino]-7,8-dihydrofurano[3,2-e][1]benzofuran-1-carboxamide;
2-(4-fluorophenyl)-5-[(2-hydroxyethyl)(methylsulfonyl)amino]-N,7-dimethyl-7,8-dihydrofurano[3,2-e][1]benzofuran-1-carboxamide;
5-bromo-2-(4-fluorophenyl)-N,7-dimethyl-7,8-dihydrofurano[3,2-e][1]benzofuran-1-carboxamide;
5-amino-2-(4-fluorophenyl)-N,7-dimethyl-7,8-dihydrofurano[3,2-e][1]benzofuran-1-carboxamide;
5-(dimethylamino)-2-(4-fluorophenyl)-N,7-dimethyl-7,8-dihydrofurano[3,2-e][1]benzofuran-1-carboxamide;
5-(1-acetylpyrrolidin-2-yl)-2-(4-fluorophenyl)-N,7-dimethyl-7,8-dihydrofurano[3,2-e][1]benzofuran-1-carboxamide;
5-(3,5-dimethylisoxazol-4-yl)-2-(4-fluorophenyl)-N,7-dimethyl-7,8-dihydrofurano[3,2-e][1]benzofuran-1-carboxamide;
2-(4-fluorophenyl)-N,7,7-trimethyl-7,8-dihydrofurano[3,2-e][1]benzofuran-1-carboxamide;
2-(4-fluorophenyl)-N,7,7-trimethyl-5-[(methylsulfonyl)amino]-7,8-dihydrofurano[3,2-e][1]benzofuran-1-carboxamide;
2-(4-fluorophenyl)-N,7,7-trimethyl-5-[methyl(methylsulfonyl)amino]-7,8-dihydrofurano[3,2-e][1]benzofuran-1-carboxamide;
2-(4-fluorophenyl)-7-(hydroxymethyl)-N-methyl-5-[methyl(methylsulfonyl)amino]-7,8-dihydrofurano[3,2-e][1]benzofuran-1-carboxamide;
2-(4-fluorophenyl)-N-methyl-5-[methyl(methylsulfonyl)amino]-7-(morpholin-4-ylmethyl)-7,8-dihydrofurano[3,2-e][1]benzofuran-1-carboxamide;
2-(4-fluorophenyl)-N-methyl-5-[methyl(methylsulfonyl)amino]-7-[(4-methylpiperazin-1-yl)methyl]-7,8-dihydrofurano[3,2-e][1]benzofuran-1-carboxamide;
2-(4-fluorophenyl)-N-methyl-5-[methyl(methylsulfonyl)amino]-7-{[methyl(phenyl)amino]methyl}-7,8-dihydrofurano[3,2-e][1]benzofuran-1-carboxamide;
2-(4-fluorophenyl)-N-methyl-5-[methyl(methylsulfonyl)amino]-7-(1H-1,2,4-triazol-1-ylmethyl)-7,8-dihydrofurano[3,2-e][1]benzofuran-1-carboxamide;
2-(4-fluorophenyl)-N-methyl-5-[methyl(methylsulfonyl)amino]-7-[(2-oxopyridin-1(2H)-yl)methyl]-7,8-dihydrofurano[3,2-e][1]benzofuran-1-carboxamide;
2-(4-fluorophenyl)-N-methyl-5-[methyl(methylsulfonyl)amino]-7-[(pyridin-2-yloxy)methyl]-7,8-dihydrofurano[3,2-e][1]benzofuran-1-carboxamide;
methyl {2-(4-fluorophenyl)-1-[(methylamino)carbonyl]-7,8-dihydrofurano[3,2-e][1]benzofuran-7-yl}acetate;
methyl {2-(4-fluorophenyl)-1-[(methylamino)carbonyl]-5-[methyl(methylsulfonyl)amino]-7,8-dihydrofurano[3,2-e][1]benzofuran-7-yl}acetate;
7-[2-(dimethylamino)-2-oxoethyl]-2-(4-fluorophenyl)-N-methyl-5-[methyl(methylsulfonyl)amino]-7,8-dihydrofurano[3,2-e][1]benzofuran-1-carboxamide; and
2-(4-fluorophenyl)-N-methyl-5-[methyl(methylsulfonyl)amino]-7-{2-[methyl(phenyl)amino]-2-oxo ethyl}-7,8-dihydrofurano[3,2-e][1]benzofuran-1-carboxamide;
and pharmaceutically acceptable salts thereof.

15. The compound of the formula (I) according to claim 1 in which:
Ar is phenyl, pyridyl, pyridazinyl, pyrimidinyl, pyrazinyl, thienyl, furanyl, pyrazolyl or imidazolyl, optionally substituted by Q¹;
Q¹ is fluorine, chlorine, bromine, hydroxy, C₁₋₄alkyl or C₁₋₄alkoxy;
R¹ is hydrogen, methyl or ethyl;
R² is hydrogen, methyl or ethyl;
R³ is hydrogen, fluoro, chloro, bromo, NR^{a}R^{b}, Het or heteroaryl, optionally substituted by methyl or C(O)CH₃;
R⁴ is hydrogen, hydroxy, NR^{c}R^{d}, heteroaryl, O-heteroaryl, C(O)C₁₋₂alkyl or C(O)NR^{c}R^{d}, optionally substituted by oxo, where R^{c} and R^{d} are independently selected from C₁₋₄alkyl or phenyl, or where R^{c} and R^{d}, together with the nitrogen atom to which they are attached, form a 6-membered heteroaliphatic ring optionally containing one O atom and/or one NH or NC₁₋₄alkyl group; and
R⁵ is hydrogen or C₁₋₂alkyl;
or a pharmaceutically acceptable salt thereof.

16. A method of inhibiting hepatitis C virus polymerase and/or of treating or preventing an illness due to hepatitis C virus, the method involving administering to a human or animal (preferably mammalian) subject suffering from the condition a therapeutically or prophylactically effective amount of the pharmaceutical composition according to claim 10, or a pharmaceutically acceptable salt thereof.
